(19) United States
(12) Patent Application Publication
(10) Pub. No.: US 2019/0300407 A1
(43) Pub. Date: Oct. 3, 2019

(54) Biological Floc and Uses Thereof, for example, in Sludge Innocent Treatment and Industrial Water Purification Treatment

(71) Applicant: KUNSHAN HUAKE INSTITUTE OF BIOPOLYMER CO., LTD, Kunshan, Suzhou, Jiangsu (CN)

(72) Inventors: Dalei GUO, Kunshan, Suzhou, Jiangsu (CN); Yongping LIU, Kunshan, Suzhou, Jiangsu (CN); Xuejun WANG, Kunshan, Suzhou, Jiangsu (CN)

(73) Assignee: Kunshan Huake Institute of Biopolymer Co., Ltd, Kunshan, Suzhou, Jiangsu (CN)

(21) Appl. No.: 16/305,853

(22) PCT Filed: May 9, 2017
(86) PCT No.: PCT/CN2017/083530

§ 371 Date: Nov. 29, 2018

(30) Foreign Application Priority Data
Oct. 25, 2016 (CN) CN201610985147.X
Apr. 21, 2017 (CN) CN201710265367.X

(51) Int. Cl. C02F 3/34; B09C 1/10; C02F 11/12; C05G 3/04; C09K 17/16; C12N 1/16; C12N 1/20; C12N 9/50; C12R 1/02; C12R 1/10; C12R 1/125

(52) U.S. Cl. CPC C02F 3/341; B09C 1/10; C02F 11/12; C05G 3/04; C09K 17/16; C12R 1/125; C12N 1/20; C12N 9/50; C12R 1/02; C12R 1/10; C12N 1/16

(57) ABSTRACT

Provided are a biological floc, a process for the preparation thereof and uses thereof in aquaculture, water purification, industrial water treatment, soil remediation and improvement, and sludge innocent treatment, wherein the biofloc comprises decomposed straw powder, a first microbial agent, a nitrifying bacteria and a denitrifying bacteria, wherein the first microbial inoculant comprises Bacillus subtilis and selective actinomycetes. The process for the preparation of the biological floc comprises firstly adding the microbial agent to the decomposed straw powder, and then successively adding the nitrifying bacteria and the denitrifying bacteria. Further provided is an organic bacterial fertilizer comprising sludge, a biological floc and fermenting bacteria.

### TECHNICAL FIELD OF THE INVENTION

The present disclosure relates to a biological floc and a process for the preparation thereof, and the biological floc is suitable for water pollution environment treatment and purification treatment of liquid emissions in aquaculture and other farming, and is also suitable for soil remediation and improvement of saline-alkali land, industrial water purification treatment.

The present disclosure also relates to the technical field of sludge innocent treatment of sewage treatment plants, in particular to the application of a biological floc in sludge innocent treatment in a sewage treatment plant, specifically to an organic bacterial fertilizer using sludge as a raw material and a process for the preparation thereof.

### BACKGROUND OF THE INVENTION

The concept of biological flocs appears in the field of aquaculture. Biological flocs in aquaculture refer to agglomerates formed by organisms mainly composed of aerobic microorganisms and inorganic substances in aquaculture waters via biological flocculation, which are mostly composed of bacteria, zooplankton and phytoplankton, organic debris and some inorganic substances. Today, Bio-floc Technology (BFT) is a new type of breeding technique to supplement the organic carbon materials by feeding a large amount of bait to the aquaculture water, to maintain a certain carbon-nitrogen ratio of the water body, to directionally adjust and control the microbial community of the culture system, and to convert ammonia nitrogen in the water into bacterial proteins by using microorganisms, and thus significantly improve feed utilization efficiency. Bio-floc technology was first proposed systematically in 1999 by the Israeli breeding expert Avnimelec, and successfully tested in Indonesia in 2005. As a technology that can maintain the stability of water environment, reduce the amount of water exchange, increase the survival rate of culture, increase the yield and reduce the feed coefficient, bio-floc technology is considered to be an effective alternative technology that solves the environmental constraint and feed cost faced in the development of aquaculture industry, which not only reduces the cost of the bait, but also solves the water quality problem in the breeding process.

It is also known that in each country, the amount of sludge discharged from sewage treatment plants is increasing year by year, and the environmental pollution problems caused by sludge are becoming increasingly prominent, which has caused great safety hazards, environmental pressures and economic burdens. The sludge contains a large amount of toxic and harmful substances such as heavy metal substances and pathogenic bacteria, and the sludge that has not been safely and environmentally treated has a greater environmental hazard. The sludge treatment process unit mainly includes processes such as sludge concentration, dehydration, digestion, fermentation, and drying. There are usually several methods for sludge treatment: (1) landfill, which means that the sludge is disposed of in a special landfill, and is flattened and compacted to cover the soil like a normal solid waste; (2) incineration, which means that disposal of sludge uses the heat of the sludge itself during the incineration process, and after the incineration, the organic matter is completely mineralized, and its own properties have completely changed. The discharge of atmospheric pollutants, fly ash and ash residue generated by sludge drying and incineration shall be treated and disposed of accordingly; (3) using in building materials, which generally includes using as cement additives, and in brick making and lightweight aggregates. In addition, the use of sludge in building materials further comprises using sludge in the manufacturing of biochemical fiberboard, roadbed materials, fences and other processes, and these technologies still have disadvantages of being premature or too small consumption capacity, and cannot meet the discharge of sludge; (4) agricultural use, which can solve a large number of sludges, and there are many kinds of sludge treatment and disposal technologies, but the technology is spotty, and there is no uniform standard to guide sludge treatment, which leads to increased costs, and even worse, it causes secondary pollution of the environment.

Although biological flocs have long been known in the field of aquaculture, there has never been a report on the application of biological flocs in the sludge innocent treatment to prepare organic bacterial fertilizers. At the same time, there is no report on formulas of biological flocs or preparation processes thereof, and no related report of organic bacterial fertilizer using biological flocs. In addition, there have been no reports of applying biological flocs to water purification treatment, soil remediation and saline-alkali and improvement and achieving excellent results.

It is also known that China's straw resources are extremely abundant, and in recent years, relevant departments at home and abroad have continuously explored the harmless treatment and resource utilization of crop straws, including direct returning to the fields, raising livestock and returning manure to the field, straw power generation and ethanol production, etc., however, which can not effectively use and treat crop straw, and have a complex process, a slow degradation rate, high cost, and even cause more serious environmental pollution. Therefore, how to quickly and effectively treat and utilize crop straw becomes a major challenge.

The invention provides a technology for preparing biological flocs by using straw resources, and further studies the application of biological flocs, in particular, the application of biological flocs in industrial water purification treatment and sludge innocuous treatment, and has achieved remarkable effects, which helps to solve the problems such as sludge innocent treatment and crop straw treatment.

### SUMMARY OF THE INVENTION

The first purpose of the present disclosure is to provide a biological floc and a process for the preparation thereof, and the biological floc of the present disclosure has low cost and is suitable for aquaculture, and is also suitable to use in water purification, soil remediation and improvement of saline-alkali land, industrial water purification treatment, sludge innocent treatment, and the like.

The second purpose of the present disclosure is to provide uses of the biological floc of the present disclosure in aquaculture, water purification, soil remediation and improvement of saline-alkali land, industrial water purification treatment, sludge innocent treatment, and the like.

The third purpose of the present disclosure is to provide an organic bacterial fertilizer prepared by taking the biological floc of the present disclosure and sludge as raw material, which has excellent manurial effect and low cost.

To achieve the above mentioned purposes, the present disclosure provides a biological floc comprising decomposed straw powder, a first microbial agent, a nitrifying bacteria and a denitrifying bacteria, wherein the first microbial agent comprises first Bacillus subtilis and selective actinomycetes.

Preferably, the first Bacillus subtilis is a combination of Bacillus subtilis KX-1 having Depository Accession Number CCTCC No. M208057, Bacillus subtilis KX-2 having Depository Accession Number CCTCC No. M208058, and Bacillus subtilis KX-4 having Depository Accession Number CCTCC No. M208060, further preferably, a mass ratio of the three is 1:(0.4-0.85):(0.5-0.85).

Preferably, the first microbial agent comprises actinomycetes. Further preferably, a mass ratio of actinomycetes to Bacillus subtilis KX-1 having Depository Accession Number CCTCC No. M208057 is (0.3-0.75): 1.

In some specific implementations of the present disclosure, the first microbial agent further comprises other beneficial microorganisms, and the other beneficial microorganisms comprise, but not limited to, lactic acid bacteria, Bacillus natto, photosynthetic bacteria, yeasts, Bacillus licheniformis, and also may be a combination thereof.

According to a specific aspect of the present disclosure, a dosage mass ratio of Bacillus subtilis KX-1 to the other beneficial microorganism is 1:(0.1-0.8), more preferably is 1:(0.5-0.8).

According to the present disclosure, a dosage of the first microbial agent is generally 5 wt % to 25 wt % of the decomposed straw powder. As a preferred embodiment, the dosage of the first microbial agent is 8 wt % to 20 wt % of the decomposed straw powder, more preferably, the dosage of the first microbial agent is 12 wt % to 20 wt % of the decomposed straw powder.

According to the present disclosure, a dosage of the nitrifying bacteria and the denitrifying bacteria is generally equal to or greater than 80 mL per kilogram of the decomposed straw powder. Preferably, the dosage of the nitrifying bacteria and the denitrifying bacteria is generally equal to or greater than 100 mL per kilogram of the decomposed straw powder. According to the present disclosure, the biological floc is generally obtained through following steps: adding the first microbial agent to the decomposed straw powder, mixing, and then successively adding the nitrifying bacteria and the denitrifying bacteria. Preferably, the biological floc has a moisture content of 10 wt % to 30% wt %.

According to the present disclosure, the decomposed straw powder is obtained by grinding fermented and decomposed straw. The decomposed straw may be obtained through the existing fermentation and decomposing methods. However, it is preferably prepared according to a process described as follows of the present disclosure, and this process is not limited by temperature, and can ensure the preparation of the decomposed straw powder under a very low temperature, with a more complete decomposition of the straw and a relatively high nutrient content, and meanwhile also reserves a lot of beneficial bacteria.

A second technical solution employed by the present disclosure is: a process of the preparation for a biological floc of the present disclosure, which comprises the following steps:

(1) preparing the decomposed straw powder: firstly, a second microbial agent is activated, then mixed with nutrients to give a mixed liquor, and then mixed with a plurality of dry straws and a selective volcanic rock biological agent to compost and decompose the straws, and finally the straws is pulverized to >80 mesh or more, the volcanic rock biological agent comprises a volcanic rock particle carrier and a third microbial agent implanted on the volcanic rock particle carrier; and

(2) adding the first microbial agent to the decomposed straw powder, and then successively adding the nitrifying bacteria and the denitrifying bacteria to obtain the biological floc. Preferabaly, the second microbial agent and the third microbial agent respectively comprise Bacillus subtilis KX-1 having Depository Accession Number CCTCC No. M208057, actinomycetes, Bacillus subtilis KX-2 having Depository Accession Number CCTCC No. M208058, and Bacillus subtilis KX-4 having Depository Accession Number CCTCC No. M208060, and a mass ratio of the four is 1:(0.3-0.75):(0.4-0.85):(0.5-0.85).

Preferably, the mass ratio of Bacillus subtilis KX-1, actinomycetes, Bacillus subtilis KX-2, and Bacillus subtilis KX-4 in the second microbial agent and the third microbial agent respectively is 1:(0.6-0.7):(0.7-0.8):(0.7-0.8).

Preferably, the second microbial agent comprises enzyme preparations such that acid proteases thereof are ≥2000 u/g, neutral proteases thereof are ≥10000 u/g, and cellulases thereof are ≥10000 u/g. Wherein, the enzyme preparations are acid proteases, neutral proteases and/or cellulases, and when enzymes in the microbial agent are insufficient, the corresponding enzyme preparations are added.

More preferably, the second microbial agent and the third microbial agent further optionally comprise other beneficial microorganisms and enzyme preparations, respectively, and the other beneficial microorganisms are selected from the group consisting of lactic acid bacteria, photosynthetic bacteria, yeasts, Bacillus licheniformis and combinations thereof.

According to a specific and preferable aspect of the present disclosure, the second microbial agent comprises the other beneficial microorganisms and a dosage mass ratio of Bacillus subtilis KX-1 to the other beneficial microorganism in the second microbial agent is 1:(0.1-0.8), more preferably is 1:(0.5-0.8).

According to another specific and preferable aspect of the present disclosure, the third microbial agent comprises the other beneficial microorganisms and a dosage mass ratio of Bacillus subtilis KX-1 to the other beneficial microorganism in the third microbial agent is 1:(0.1-0.8), more preferably is 1:(0.5-0.8).

Preferably, the volcanic rock biological agent is prepared through following steps:

1) grinding volcanic rocks into volcanic rock particles of 20-40 mesh, removing volcanic rock powder to obtain a volcanic rock particle carrier;

2) preparing a bacteria solution by the third microbial agent and water according to a dosage mass ratio of 1:(20-50), then adding nitrogen source and carbon source to activate strains, and immersing the volcanic rock particle carrier in the bacteria solution for 24-72 h when more than 80% of Bacillus in the bacteria solution is in a vegetative state and viable count is >5 billion/mL, and then taking out and draining the volcanic rock particle carrier to give the volcanic rock biological agent.

According to a preferred aspect of the present disclosure, in the step (1), a dosage mass ratio of the second microbial agent and the dry straws is 1:(1000-2000).

According to another preferred aspect of the present disclosure, in the step (1), a dosage mass ratio of the second microbial agent, the volcanic rock biological agent and the dry straws is 1:(50-80):(1000-2000).

In the present disclosure, the bacterial strain may be activated by using conventional methods.

Preferably, the nutrient substances comprise urea and amino acid particles.

Preferably, when the straws are subjected to composting, fermentation and decomposition, the moisture is controlled to be 50-65%.

Preferably, during composting of the straws, vent tubes are planted, then plastic film is covered on the compost pile, and the plastic film covered on the vent tubes is removed when a temperature of the compost pile rises up to 50-60° C. and the fermentation continues for 10-15 days, then the compost pile is turned over and replenished with water, and continues to ferment for 5-15 days until fermentation is matured.

A third technical solution employed by the present disclosure is: use of the biological floc of the present disclosure mentioned above in aquaculture, water purification, industrial water treatment, soil remediation and improvement and sludge innocent treatment for preparing fertilizer.

The biological floc of the present disclosure can be used for aquaculture, and if other engineering bacteria are added, it is still a very good bioremediation material, such as for soil remediation and saline-alkali improvement. In addition, the biological floc can also be used as a bio-filler in industrial water treatment MBR, to replace activated sludge and reduce sludge volume.

Specifically, the biological floc is placed in the river to effectively treat the river water. In industrial water treatment, biological floc can replace existing biological flocculant and activated sludge to effectively remove ammonia nitrogen.

A fourth technical solution employed by the present disclosure is: an organic bacterial fertilizer, a raw material formula thereof comprising sludge, the biological floc of the present disclosure, and fermenting bacteria.

Further, the fermenting bacteria comprises a second Bacillus subtilis. In a specific implementation, the fermenting bacteria is a second Bacillus subtilis.

According to a preferred aspect of the present disclosure, the second Bacillus subtilis is selected from the group consisting of Bacillus subtilis KX-1 having Depository Accession Number CCTCC No. M208057, Bacillus subtilis KX-2 having Depository Accession Number CCTCC No. M208058, Bacillus subtilis KX-4 having Depository Accession Number CCTCC No. M208060 and combinations thereof.

In some specific implementations of the present disclosure, the second Bacillus subtilis is a combination of Bacillus subtilis KX-1 having Depository Accession Number CCTCC No. M208057, Bacillus subtilis KX-2 having Depository Accession Number CCTCC No. M208058, Bacillus subtilis KX-4 having Depository Accession Number CCTCC No. M208060, and a mass ratio of the three is 1:(0.4-0.85):(0.5-0.85).

The present disclosure defines the “first Bacillus subtilis” and the “second Bacillus subtilis” just for distinguishing the Bacillus subtilis respectively comprised in the biological floc and the organic bacterial fertilizer, and the “first Bacillus subtilis” and the “second Bacillus subtilis” may be the same and may be different.

According to the present disclosure, the sludge comprises aged sludge and/or dried sludge from a municipal sewage treatment plant having a mass ratio greater than or equal to 70%. The sludge is preferably activated sludge. More preferably, the sludge does not contain flocculating agents or coagulant aids, and all kinds of heavy metal indexes thereof meet the national standard NY884-2012.

According to the present disclosure, a dosage mass ratio of the biological floc to the sludge is 1:(1-20), more preferably 1:(3-10), and further preferably 1:(5-10).

According to the present disclosure, considering only fertilizer efficiency, the weight ratio of the fermenting bacteria (dry bacteria) to the sludge can be greater than or equal to 0.01%. Overall considering the cost, the weight ratio of the fermenting bacteria (dry bacteria) to the sludge is preferably less than or equal to 5%, more preferably less than or equal to 1%, and most preferably less than or equal to 0.5%.

Preferably, the weight ratio of the fermenting bacteria (dry bacteria) to the sludge is 0.01%-1%. More preferably, the weight ratio of the fermenting bacteria (dry bacteria) to the sludge is 0.05%-0.5%.

In some preferred implementations of the present disclosure, the organic bacterial fertilizer is a fermentation product obtained by mixing, composting and fermenting the sludge, the biological floc and the fermenting bacteria, or a mixture of the fermentation product and other fertilizer additives. The other fertilizer additives may be those commonly used in the art. According to a specific aspect of the present disclosure, the other fertilizer additives comprise a sustained release agent, and the prepared organic bacterial fertilizer is sustained release fertilizer.

A fifth technical solution employed by the present disclosure is: a process of the preparation for the organic bacterial fertilizer of the present disclosure mentioned above, and the process comprises:

step (1): composting and fermenting

mixing and evenly stirring the biological floc, the sludge and the fermenting bacteria to form a mixed material, and stacking the mixed material into a cone or a strip-shaped trapezoid, covering a surface layer of the mixed material with a film and arranging an aeration pipe at a bottom of the mixed material to start composting and fermenting, and aerating inside a pile body using the aeration pipe during the composting and fermenting process;

step (2): turning over and replenishing water

after a core temperature of the mixed material rises up to a highest point and continues for 1-5 days, turning over the mixed material and replenishing water;

step (3): re-fermenting

composting and fermenting the mixed material processed through the step (2) once more until fermentation is matured;

step (4): granulating

granulating and shaping the mixed material fermented once again in the step (3), to give granular organic bacterial fertilizer.

According to the present disclosure, the above mentioned “highest point” means an upper temperature limit that the temperature of the mixed material can reach during composting and fermenting.

Further, in step (3), the core temperature of the pile of the mixed material firstly rises up and then drops, and fermentation is matured when a difference between the core temperature and a surface temperature of the mixed material is within ±5° C.

Further, step (4) is carried out directly after step (3), or step (4) is carried out after mixing a product of step (3) with other fertilizer additives.

According to a preferred aspect of the present disclosure, the other fertilizer additives comprise a sustained release agent, and the prepared organic bacterial fertilizer is sustained release fertilizer.

All of the bacteria used in the present disclosure, including Bacillus subtilis, are known. Wherein the depository sites of Bacillus subtilis KX-1, Bacillus subtilis KX-2 and Bacillus subtilis KX-4 is the China Center for Type Culture Collection.

The first microbial agent, the second microbial agent and the third microbial agent of the present disclosure do not contain nitrifying bacteria and denitrifying bacteria.

According to the present disclosure, the first microbial agent, the second microbial agent and the third microbial agent may be the same or may be not. In a specific implementations of the present disclosure, they are the same.

According to the present disclosure, the microbial agents may be a mixture of several materials above mentioned; may also be several material separately packaged that are mixed according to a ratio when use. Preferably, the microbial agents may be a mixture of several materials above mentioned.

The straw of the present disclosure may be straw of any crop, in particular, corn straw, rice straw and the like.

The present disclosure also relates to use of the biological floc mentioned above in sludge innocent treatment to prepare fertilizer. Preferably, the fertilizer is an organic fertilizer. A specific process comprises, for example, mixing, composting and fermenting the biological floc, the sludge and the fermenting bacteria.

The present disclosure applies the biological floc in waste sludge from a municipal sewage treatment plant, and innocently treats it to prepare organic fertilizer, which can quickly solve the problems of accumulation, pollution and waste of agricultural waste, and can realize the recycling of resources. In the agricultural production, vigorously promoting the application of organic fertilizer, with little or no chemical fertilizer to the greatest extent, it will effectively promote the sustainable development of agriculture and realize the harmless treatment and resource utilization of agricultural and industrial waste.

The use of biological floc to treat sludge into organic fertilizer reduces environmental pollution, prevents organic waste from polluting the environment, is conducive to improving urban and rural ecological environment, and is beneficial to human health, and on the other hand, organic fertilizer being applied to the soil instead of part of chemical fertilizers improves the soil structure, increases soil nutrient content, improves soil biological activity, plays a role in nutrient returning, fertilizing soil and improving crop quality, and increasing the output of agricultural products, effectively promotes farmers' cost reduction and income increase, and reduces the investment in the crop industry, which finally plays a positive role in promoting the development of ecological agriculture and organic agriculture.

Due to the implementation of the above technical solutions, the present disclosure has the following advantages over the prior art:

The present disclosure provides a formula of a biological floc, which is mainly composed of decomposed straw powder, and the main technical indexes thereof are equal to and even better than foreign biological floc of the same type by adding specific microbial agents, nitrifying bacteria and denitrifying bacteria, and the experiment proves that the biological floc is very effective in purifying the river sewage.

In the process of the preparation for the biological floc of the present disclosure, in the preparation of the decomposed straw powder, low-temperature bacteria serves as the induction bacteria, which contains a plurality of complex functional beneficial microorganisms mainly composed of Bacillus subtilis (including Bacillus natto) and actinomycetes, and can breed under low temperature conditions and promote the high-speed reproduction of other bacterial agents. It can not only fertilize at high temperature, but also can fertilize at low temperature, and it is not restricted by regions and seasons, and can be used in all seasons, so that farmers can handle straw in the vicinity of farmland, and realize the complete decomposition of straw locally, without carrying the straw, and saving manpower, material resources and financial resources; the present disclosure further combines the volcanic rock abundant in Jilin Province, utilizes the structure properties of porousness and large surface area of the volcanic rock, exerts its effects in water retention, serving as a carrier of the microbial agent and enhancing the heat dissipation function, and solves the problem of utilization of natural mineral resources.

The organic bacterial fertilizer provided by the present disclosure is mainly composed of sludge, is obtained by adding the biological floc of the present disclosure and mixing with a fermenting agent, has significantly better fertilizer efficiency than the organic fertilizer currently sold on the market, has a wide range of raw material sources and low cost, and especially solves the problem of innocent treatment of sludge and disposal of waste straw resources.

### BRIEF DESCRIPTION OF THE DRAWINGS

FIG. 1 is a picture of decomposed straw powder prepared in Embodiment 1;

FIG. 2 is a picture of the biological floc prepared in Embodiment 2;

FIG. 3 is a picture of sludge from a municipal sewage treatment plant;

FIG. 4 shows changing curves of a core temperature and a surface temperature of the organic bacterial fertilizer of the present disclosure during composting;

FIG. 5 is a state diagram of the mixed material during turning over the pile;

FIG. 6 shows a specific shape of the granular organic bacterial fertilizer after granulating and shaping.

### DETAILED DESCRIPTION OF EXEMPLARY EMBODIMENTS

The biological floc of the present disclosure employs the crop straw as raw material, and is a new natural biological material produced by adding specific microorganism strains and selectively combines with volcanic rock fine particles of Changbai Mountain through bioengineering (deep fermentation).

The biological floc of the present disclosure not only can make full use of the straw which has being waste in the past, to turn waste into wealth, but also reduces the pollution caused by burning straw to the air. In the fermentation process, under the deep effect of the beneficial low temperature bacteria, the straw is thoroughly matured such that the product has extremely high nutrient content, and also stores a large number of beneficial bacteria. The matured straw is subjected to subsequent processing into a fine powder of 80 mesh or more, preferably 80-100 mesh, to form a high-quality biological skeleton, and then appropriately added with bacteria such as Bacillus subtilis, lactic acid bacteria, Bacillus natto and the like, which are added during fermentation, and then sprayed with nitrifying bacteria and denitrifying bacteria successively, and controlled in a moisture between 10-30%, to give the biological floc. The biological floc can rapidly form a biofilm in the water body, fully decomposes the organic matter and ammonia nitrogen in the water to convert into nitrogen finally, thereby maintaining a stable water quality of the aquaculture water body.

The beneficial bacteria added in the floc can effectively improve the intestinal tract of aquaculture, help digestion and absorption, enhance immunity and reduce the bait coefficient.

Due to the microorganisms completely decompose the degradable material of the straw during the pre-fermentation process of the biological floc, therefore, when the biological floc is released into the water body, it does not increase the load, does not pollute the water body, does not cause secondary pollution, and can purify the water body through the contained beneficial bacteria.

If other engineering bacteria are added, it is still a very good bioremediation material, such as for soil remediation and saline-alkali improvement, and meanwhile, it also can be used as a bio-filler in industrial water treatment MBR, to replace activated sludge and reduce sludge volume.

In the same time, the present disclosure further presents that the biological floc is combined with the sludge to prepare fertilizer. Sludge is a sediment of urban sewage, and the sludge contains nutrients necessary for plant growth and rich organic matter, has a good effect of improving physical and chemical properties of soil, is the main energy and nutrient sources of soil microorganisms, is an important measure to reduce agricultural costs, is an important link to maintain and promote soil nutrient balance and fertilize soil, and can significantly increase soil organic matter content, can significantly reduce nitrogen leaching in soil, weakens pollution of groundwater by inorganic chemical fertilizers, and has promoting effect on the adjustment of water, fertilizer, gas and heat conditions and the abilities of fertilizer maintenance and water retention of soil, and especially for soils with poor structure, it is a rare modifier. Fermenting sludge into organic fertilizer can not only reduce urban pollution, recycle waste and apply it into farmland, but also can fertilize soil and improve the quality of agricultural products, which achieves many things at one stroke The present disclosure will be further described below in combination with specific embodiments, but the present disclosure is not limited to the following embodiments.

### Embodiment 1: Preparation of Decomposed Straw

I. Main Raw Material

1. A second microbial agent, according to a mass ratio of:

Bacillus subtilis KX-1 having Depository Accession Number CCTCC No. M208057 25%

Actinomycetes 15%

Bacillus subtilis KX-2 having Depository Accession Number CCTCC No. M208058 20%

Bacillus subtilis KX-4 having Depository Accession Number CCTCC No. M208060 20%

Other beneficial microorganisms such as lactic acid bacteria and PSB (photosynthetic bacteria) 20%

The prepared complex bacterial agent requires: acid protease ≥2000 u/g, neutral protease >10000 u/g, cellulase ≥10000 u/g, and the insufficient part is satisfied by adding corresponding enzyme preparations.

Bacillus subtilis KX-1 may be survive and breed in 2-8° C.

2. A Volcanic Rock Biological Agent

The volcanic rock biological agent takes advantages of structure properties of porousness and large surface area, containing of a large number of dozens kinds of minerals such as silicon, potassium, sodium, iron, magnesium, aluminum, silicon, calcium, titanium, manganese, iron, nickel and cobalt, and trace elements, as well as the chelation of heavy metals of volcanic rocks, and is a product formed by using volcanic rocks as a carrier, and then implanting a variety of beneficial microbial flora (including various benifical microorganism such as actinomycetes, Bacillus, etc., mainly low temperature bacteria) that are proven to be harmless to humans and animals. The volcanic rock biological agent has the functions of microbial enrichment and slow release in the composting process and the effect of heat dissipation and ventilation, and moreover, it can retain water and store water, promote the formation of soil aggregated structure, effectively improve the microbial environment of plant roots, and provide trace elements for crop growth, and promote plant growth.

The specific process of the preparation:

a, Preparation of base material

Natural volcanic rocks were ground into particles smaller than 1 mm, and then powder was removed, for use in a later step;

b, Preparation of bacteria solution

A third microbial agent was prepared according to the following fomula (weight ratio):

Bacillus subtilis KX-1 having Depository Accession Number CCTCC No. M208057 25%

Actinomycetes 15%

Bacillus subtilis KX-2 having Depository Accession Number CCTCC No. M208058 20%

Bacillus subtilis KX-4 having Depository Accession Number CCTCC No. M208060 20%

Other beneficial microorganisms such as Lactobacillus and PSB 20%.

\textcircled{1} Activation

The third microbial agent was placed in a clean square tank with water inlet, aeration, heating temperature control and drainage functions, diluted according to 1:(20-50), added with a certain amount of nitrogen source and carbon source to activate the strain;

\textcircled{2} Immersing

the treated carrier was placed and immersed in a square tank after most of Bacillus was in the vegetative state via microscopic examination, and the number of viable bacteria was determined by the plate colony counting >5 billion/mL;

\textcircled{3} Draining and Drying

After immersing for 24-72 h, the bacterial solution was discharged from the bottom, and the carrier was tacking out from the tank and drained off.

3. Amino Acid Particles

The amino acid particles were prepared by pure biological fermentation and concentration, and were rich in nitrogen and other nutrients.

II. Preparation Process

(1) Related Equipments and Materials

1. Large-scale feed crushing and cutting machine: for cutting the dried straw into a length of 1-2 cm.

2. Large-scale stirrer: large-scale mortar mixer of front feeding and rear discharging type.

3, Large-scale sprayer: for spraying the mixed liquid of bacteria and nutrient solution when stirring.

4. Agricultural plastic film: several.

5. Strain activation equipment.

6. Mixing related tools or equipment.

7. Vent tube: plastic tube with a diameter of about 10 cm and perforated around.

8. Other materials: brown sugar, rice bran, urea

(2) Usage, Dosage and Fermentation Cycle

1. Dosage

For per 1 Kg of the second microbial agent+60 Kg of the volcanic rock biological agent, about 1-2 tons of dry straws were fermented and decomposed, under an ambient temperature of 5-10° C. during fermentation and decomposing.

2. Usage (to Activate 1 kg of Strain and Ferment 1 T of Straws as an Example)

1) Strain Activation

It was diluted with water in a ratio of 1:(20-30), added with 4 Kg of fresh rice bran and 0.5 Kg of brown sugar, and activated for 12 h.

2) Nutrition Sources and Auxiliary Material Preparation

22 Kg of urea and 10 Kg of amino acid particles were fully dissolved in water, and diluted to about 1.2-1.5 T.

3) Mixing

The activated bacterial solution in 1) was added into the solution in 2) and mixed well to form a mixed solution.

4) Stirring: The Moisture was Controlled at about 50-65%

The pulverized straws were fed from the front end (feed port) of the stirrer, and after adding the volcanic rock biological agent, the mixed solution in 3) was simultaneously sprayed from the both ends to the feed port and the discharge port by a sprayer, and the mixture was uniformly stirred and output from the rear end of the stirrer to compost.

5) Material Piling

Depending on the actual situation, a cone (with a bottom diameter of about 3 m) or a strip-shaped trapezoid (with a bottom width of 2 m, a height of 1.5 m, and a top width of 1.0 m) was piled, and vent tubes were placed and the film is covered to ferment. The film covered on the vent tubes were removed when the temperature rised up to 50-60° C. after 3 days.

6) Turning Over for the Second Time

After composting for about 15 days, the pile was turned over and replenished with water for the second time.

3. Fermentation Cycle

About 30 days or so, the whole decomposing process was completed, and the straw became brown or dark brown; the pile had a bio-fermented scent of earthy smell and ammonia smell; the pile was covered with a large amount of white hyphae and had a dispersed texture.

See Table 1 for the composition analysis of the decomposed straw at the end of fermentation.

TABLE 1

| | Name | |
|---|---|---|
| Item | Dry rice straw | Rice straw after composting |
| Moisture (%) | 9 | 62 |
| Organic matter (g/Kg) | 53.96 | 358.77 |
| Total nitrogen (g/Kg) | 4.3 | 15.22 |
| Total phosphorus (g/Kg) | 1.87 | 2.69 |
| Total potassium (g/Kg) | 7.9 | 18.49 |

### Embodiment 2: Preparation of Decomposed Straw

The present embodiment was substantially the same with Embodiment 1, by differing in that:

1. Microbial agent, according to a mass ratio of:

Actinomycetes 40%

Bacillus subtilis KX-2 having Depository Accession Number CCTCC No. M208058 20%

Bacillus subtilis KX-4 having Depository Accession Number CCTCC No. M208060 20%

Other beneficial microorganisms such as Lactobacillus and PSB 20%.

The prepared complex bacterial agent requires: acid protease ≥2000 u/g, neutral protease ≥10000 u/g, cellulase ≥10000 u/g, and the insufficient part was satisfied by adding corresponding enzyme preparations.

2. The bacterial agent implanted in the volcanic rock biological agent was:

Actinomycetes 40%

Bacillus subtilis KX-2 having Depository Accession Number CCTCC No. M208058 20%

Bacillus subtilis KX-4 having Depository Accession Number CCTCC No. M208060 20%

Other beneficial microorganisms such as lactic acid bacteria and PSB 20%.

See Table 2 for the composition analysis of the decomposed straw after fermenting the dry straw according to the method of Embodiment 1.

TABLE 2

| | Name | |
|---|---|---|
| Item | Dry rice straw | Rice straw after composting |
| Moisture (%) | 9 | 59 |
| Organic matter (g/Kg) | 53.96 | 143.73 |
| Total nitrogen (g/Kg) | 4.3 | 10.09 |
| Total phosphorus (g/Kg) | 1.87 | 2.09 |
| Total potassium (g/Kg) | 7.9 | 9.11 |

See Table 3 for the results of comparing straw decomposition maturity of Embodiment 1 with Embodiment 2 (weight loss rate method).

TABLE 3

| | Name |
|---|---|
| Item | Weight loss rate of the straw |
| Embodiment 1 | 51.2% |
| Embodiment 2 | 37.5% |
| Note | If the difference between the weight loss rates of the |
| | straw treated by different decomposing bacterial agents |
| | in the same period of time is more than 5%, it indicates |
| | that there is a difference between the decomposition |
| | results of different decomposed bacterial products, and |
| | the decomposition results of Embodiment 1 is obviously |
| | better. |

The above test was conducted in Changchun, northeast China, at the end of May and in early June. Because the rice in Northeast China is only planted for one season, it is generally harvested every October. The test time was similar in weather, temperature and humidity to the mature and harvesting season of Jilin rice, which makes the test results more convincing.

The biological floc preparation includes the following steps:

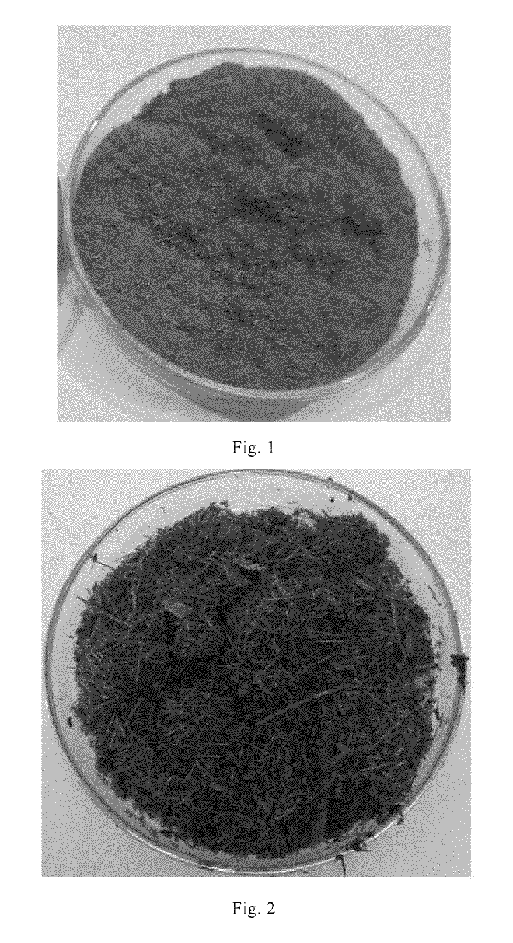

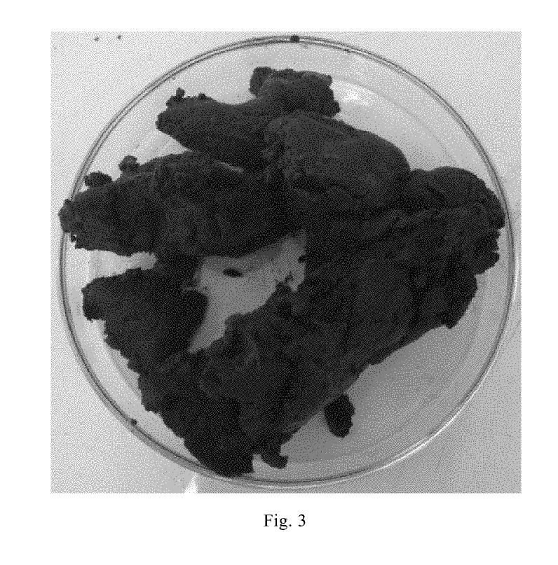

(1) The decomposed straw obtained according to Embodiment 1 was ground into powder of about 80 mesh by a grinder, to give decomposed straw powder (as shown in FIG. 1);

(2) The decomposed straw powder was added with a first microbial agent (the same with the second microbial agent in Embodiment 1) according to a ratio of 200 g of the first microbial agent per 1 Kg of the decomposed straw powder, and then sprayed with nitrifying bacteria and denitrifying bacteria successively according to a ratio of 100 mL of nitrifying bacteria and denitrifying bacteria per 1 Kg of the decomposed straw powder, to give a biological floc (as shown in FIG. 2).

The organic indexes of the biological floc obtained in this embodiment and the biological floc of a foreign company were tested, and the results are shown in Table 4, and in addition, the trace elements of the biological floc in this embodiment were tested, and the results are shown in Table 5.

TABLE 4

| | Test Methods | | | | | |
|---|---|---|---|---|---|---|
| | Moisture - drying and weighting method, organic carbon - potassium | | | | | |
| | dichromate volumetric dilution-heating method, organic matter - | | | | | |
| | potassium dichromate volumetric dilution-heating method, available | | | | | |
| | phosphorus - sodium bicarbonate extraction method, total nitrogen - | | | | | |
| | semi-micro Kjeldahl method | | | | | |
| | Test items | | | | | |
| | | Organic | Organic | Available | Total | Beneficial |
| | | carbon | matter | phosphorus | nitrogen | microorganisms |
| Classification | Moisture % | g/kg | g/kg | mg/kg | g/kg | 10⁷ |
| Biological floc | 11.3 | 471.74 | 813.28 | 456.71 | 11.45 | 273 |
| of the present | | | | | | |
| disclosure | | | | | | |
| One foreign | 28.6 | 415.57 | 716.44 | 340.51 | 3.14 | 20 |
| biological floc | | | | | | |

TABLE 5

| | Test items (g/kg) | | | | | | | | | |
|---|---|---|---|---|---|---|---|---|---|---|
| | Element | | | | | | | | | |
| | C | O | Na | Mg | Al | Si | K | Ca | Ti | Fe |
| Biological | 116.8 | 2274.4 | 114 | 58.8 | 373.2 | 727.2 | 29.2 | 166.4 | 19.6 | 120.4 |
| floc | | | | | | | | | | |

### Embodiment 4: Preparation of Organic Bacterial Fertilizer

This embodiment provided an organic bacterial fertilizer, the raw material formula thereof comprising: 1 part by weight of the biological floc, 5 parts by weight of sludge and 0.25 part by weight of fermenting bacteria (dry bacteria); wherein the biological floc was obtained according to the method of Embodiment 2; further wherein the sludge was taken from aged sludge from a municipal sewage treatment plant (the municipal sewage treatment plant mainly treats sanitary sewage (>70%), and the basic requirements for the activated sludge of the treated sanitary sewage are: 1, the activated sludge is selected from aged sludge discharged from aeration tank, and was not added with coagulant aids such as PAC and PAM before filter pressing and drying; 2, the sludge is tested for heavy metals, and the content of various heavy metals cannot exceed the national standard NY884-2012); further wherein, the fermenting bacteria is a mixed bacteria composed of Bacillus subtilis KX-1 having Depository Accession Number CCTCC No. M208057, Bacillus subtilis KX-2 having Depository Accession Number CCTCC No. M208058, Bacillus subtilis KX-4 having Depository Accession Number CCTCC No. M208060 according to a mass ratio of 1:0.8:0.8.

### Embodiment 4: Preparation of Organic Bacterial Fertilizer

Step (1): the biological floc, the sludge and the fermenting bacteria were mixed and evenly stirred to form a mixed material, and the mixed material was stacked into a cone or a strip-shaped trapezoid, a surface layer of the mixed material was covered with a film and arranged with an aeration pipe at a bottom of the mixed material to start composting and fermenting, and air was aerated inside a pile body using the aeration pipe during the composting and fermenting process;

Step (2): after a core temperature of the mixed material rose up to a highest point and continued for 2 days, the mixed material was turned over and replenished with water;

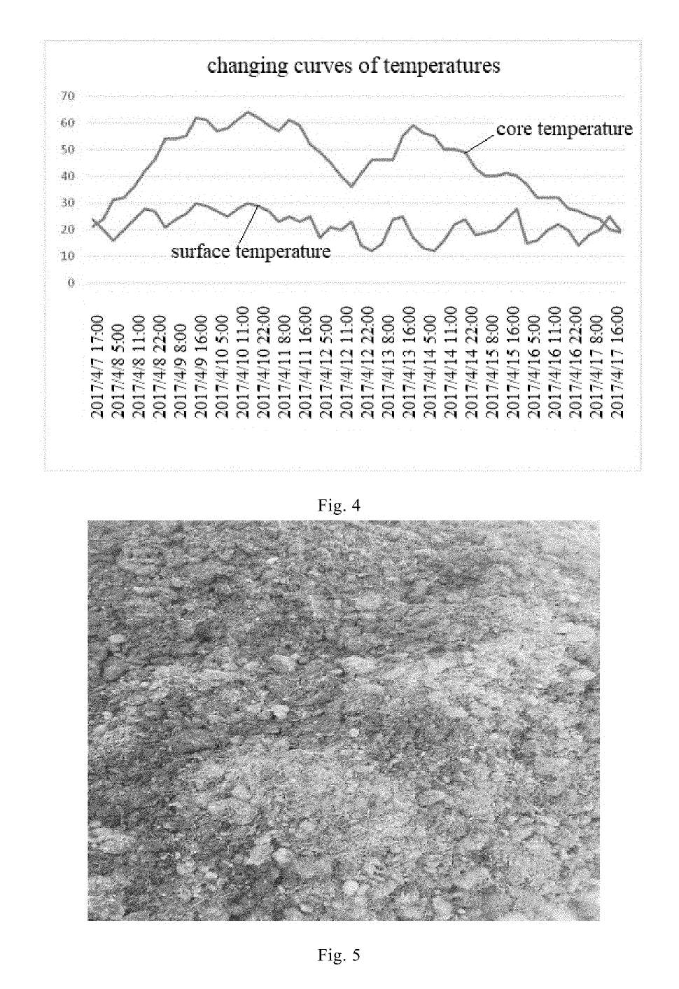

Step (3): the mixed material processed through step (2) was composted and fermented once more until fermentation is matured (refer FIG. 4 for changing curves of the core temperature and the surface temperature); the core temperature of the mixed material firstly rose up and then dropped, and a difference between the core temperature and the surface temperature of the mixed material being within 2° C. indicated that the fermentation was matured.

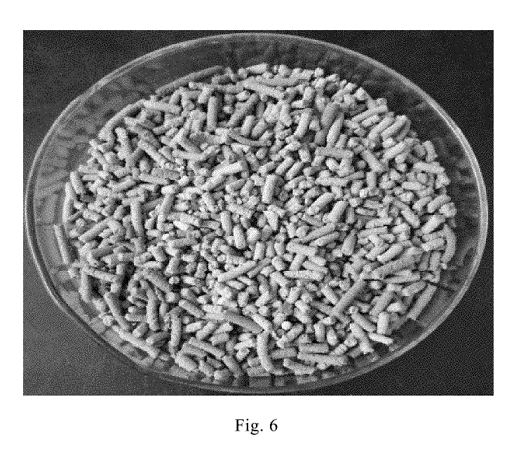

Step (4): the mixed material fermented once again in step (3) was granulated and shaped, to give granular organic bacterial fertilizer (refer to FIG. 6 for the granular shape, but it not limited to this granular shape, and may be other shapes, such as spheroid).

### Embodiment 5: Preparation of Organic Bacterial Fertilizer

This embodiment provided an organic bacterial fertilizer, the raw material formula thereof comprising: 1 part by weight of the biological floc; 10 parts by weight of sludge; and 0.01 part by weight of fermenting bacteria. The biological floc, the sludge and the fermenting bacteria were the same with those of Embodiment 4.

### Embodiment 4: Preparation of Organic Bacterial Fertilizer

Step (1): the biological floc, the sludge and the fermenting bacteria were mixed and evenly stirred to form a mixed material, and the mixed material was stacked into a cone or a strip-shaped trapezoid, a surface layer of the mixed material was covered with a film and arranged with an aeration pipe at a bottom of the mixed material to start composting and fermenting, and air was aerated inside a pile body using the aeration pipe during the composting and fermenting process;

Step (2): after a core temperature of the mixed material rose up to a highest point and continued for 3 days, the mixed material was turned over and replenished with water, and after opening the pile, there was a strong ammonia smell, and the pile was covered with a large amount of white hyphae, a photo thereof is shown in FIG. 5.

Step (3): the mixed material processed through step (2) was composted and fermented once more until fermentation is matured (refer FIG. 4 for changing curves of the core temperature and the surface temperature); the core temperature of the mixed material firstly rose up and then dropped, and a difference between the core temperature and the surface temperature of the mixed material being within 4° C. indicated that the fermentation was matured.

Step (4): the mixed material fermented once again in step (3) was granulated and shaped, to give granular organic bacterial fertilizer.

### Embodiment 6: Preparation of Organic Bacterial Fertilizer

This embodiment provided an organic bacterial fertilizer, the raw material formula thereof comprising: 1 part by weight of the biological floc; 20 parts by weight of sludge; and 0.1 part by weight of fermenting bacteria. The biological floc, the sludge and the fermenting bacteria were the same with those of Embodiment 4. The process of the preparation is the same with Embodiment 4.

### Embodiment 7: Preparation of Organic Bacterial Fertilizer

This embodiment provided an organic bacterial fertilizer, the raw material formula thereof comprising: 1 part by weight of the biological floc; 5 parts by weight of sludge; and 0.0025 part by weight of fermenting bacteria. The biological floc, the sludge and the fermenting bacteria were the same with those of Embodiment 4. The process of the preparation is the same with Embodiment 4.

Cost Analysis of the Organic Bacterial Fertilizer of the Present Disclosure

In the present application, the raw materials of the organic bacterial fertilizer are the biological floc prepared by the straw collected in the agricultural production and the sludge of the municipal sewage treatment plant, the preparation process is simple, the equipment requirements are low, and the preparation cost of the organic bacterial fertilizer is about ⅕ of the price of the domestic bio-organic fertilizer (about 2,000-3,000 yuan per ton), and is about 1/10 of the current domestic organic bacterial fertilizer (about 10,000 yuan per ton), and when compared with slow-release fertilizer, the organic bacterial fertilizer of this application can also be prepared as an intermediate product, and the yield will be greater. Therefore, our technology has an advantage in terms of time and production cost and price.

Comparison of Fertilizer Efficiency Detection Between the Organic Bacterial Fertilizer of the Present Disclosure and Commercially Available Fertilizer

The organic bacterial fertilizers of Embodiments 4 and 7 and commercially available common fertilizers are tested for moisture, organic carbon, organic matter, available phosphorus, total nitrogen and beneficial bacteria, wherein moisture—drying and weighting method, organic carbon—potassium dichromate volumetric dilution-heating method, organic matter-potassium dichromate volumetric dilution-heating method, available phosphorus—sodium bicarbonate extraction method, total nitrogen—semi-micro Kjeldahlmethod. See Table 6 for the results. In Table 6, the purchased fertilizer 1 is organic fertilizer from Jingzhong Qingxin; the purchased fertilizer 2 is organic fertilizer from Qingdao Diendi; the purchased fertilizer 3 is organic fertilizer from Jilin Wuleyuan; Fertilizer 4 is organic fertilizer from Zhongnong Lukang; purchased fertilizer 5 is organic fertilizer from Shandong Seaweed; and purchased fertilizer 6 is from Beijing Bacterial Fertilizer.

TABLE 6

| | Purchased | Purchased | Purchased | Purchased | Purchased | Purchased | | |
|---|---|---|---|---|---|---|---|---|
| Classification | fertilizer 1 | fertilizer 2 | fertilizer 3 | fertilizer 4 | fertilizer 5 | fertilizer 6 | Embodiment 4 | Embodiment 7 |
| Moisture % | 25.2 | 25.4 | 26.2 | 15.1 | 12.4 | 4.6 | 20 | 18 |
| Organic | 250.97 | 154.28 | 112.42 | 54.89 | 143.82 | 129.61 | 295.45 | 267.66 |
| carbon | | | | | | | | |
| g/kg | | | | | | | | |
| Organic | 432.67 | 265.98 | 193.82 | 94.63 | 247.95 | 223.45 | 511.08 | 489.89 |
| matter | | | | | | | | |
| g/kg | | | | | | | | |
| Total nitrogen | 4.65 | 27.87 | 9.07 | 2.68 | 70.4 | 4.88 | 29.3 | 17.4 |
| g/kg | | | | | | | | |
| Available | 129.75 | 26246.7 | 10750.76 | 363.98 | 15541.12 | 1517.22 | 15334.47 | 15567.9 |
| phosphorus | | | | | | | | |
| mg/kg | | | | | | | | |
| Beneficial | | | | | | 2.6 × 10⁷ | 6.7 × 10⁷ | 6.5 × 10⁷ |
| microorganis | | | | | | | | |
| ms | | | | | | | | |
| CFU/g | | | | | | | | |

From the comparison of Table 6, it can be found that from the analysis of nutrient composition, the organic bacterial fertilizer of the present application has obvious advantages compared with the existing six organic fertilizers on the market.

In summary, using the biological floc to make sludge into bio-organic fertilizers has the advantages of fast fermentation, good fermentation, high-temperature fermentation, low-temperature fermentation, greatly shortening production time, reducing costs, etc., which fully conforms to the sludge treatment requirements of China's national conditions, implements of sludge innocent treatment and sludge resource utilization, increases economic efficiency and avoids more serious secondary pollution.

### Embodiment 8: Test of Applying the Biological Floc to River Sewage Purification Treatment

Test water: sewage in the river dam of No. 10, Kunshan Tianrui Environmental Company,

Jiangsu Province, China (dissolved oxygen 0.62 mg/L, ammonia nitrogen 14.9 mg/L)

Water sample: 2 L

Test: Two tests were carried out successively, and the results are shown in Tables 7 and 8, respectively.

TABLE 7
First Test (Oct. 11-Oct. 12, 2016)

| | 24 h test | | 48 h test | |
|---|---|---|---|---|
| | Dissolved | Ammonia | Dissolved | Ammonia |
| Project Name | oxygen | nitrogen | oxygen | nitrogen |
| Raw water + biological | 2.82 | 5.71 | 3.35 | 2.74 |
| floc 10 ppm | | | | |
| Raw water + biological | 2.66 | 5.84 | 3.16 | 2.44 |
| floc 20 ppm | | | | |
| Raw water + biological | 2.13 | 5.53 | 3.14 | 2.12 |
| floc 40 ppm | | | | |
| Raw water + biological | 1.93 | 5.42 | 2.68 | 2.05 |
| floc 60 ppm | | | | |
| Raw water + poly | 3.20 | 6.4 | 4.35 | 3.8 |
| aluminum chloride | | | | |
| 40 ppm | | | | |

| | | |
|---|---|---|
| Maximum | 64% | 86% |
| ammonium-nitrogen | | |
| removal rate | | |

TABLE 8
Second Test (Oct. 13-Oct. 15, 2016)

| | 24 h test | | 48 h test | |
|---|---|---|---|---|
| | Dissolved | Ammonia | Dissolved | Ammonia |
| Project Name | oxygen | nitrogen | oxygen | nitrogen |
| Raw water | 4.63 | 12.6 | 4.51 | 10.6 |
| Raw water + poly | 4.20 | 5.4 | 3.35 | 2.8 |
| aluminum chloride | | | | |
| 40 ppm | | | | |
| Raw water + biological | 3.50 | 4.44 | 4.23 | 2.34 |
| floc 10 ppm | | | | |
| Raw water + biological | 3.32 | 3.99 | 4.21 | 2.29 |
| floc 20 ppm | | | | |
| Raw water + biological | 2.22 | 3.63 | 3.74 | 1.63 |
| floc 40 ppm | | | | |
| Raw water + biological | 1.78 | 3.63 | 3.76 | 1.51 |
| floc 60 ppm | | | | |

| | | |
|---|---|---|
| Maximum | 71% | 86% |
| ammonium-nitrogen | | |
| removal rate | | |

Note:

1. Take the same river water for each test, 2000 mL per unit.

2. The water temperature was the same as the outdoor water temperature.

3. The amount of aeration was the same for each test.

4. Poly aluminum chloride in the test was as a control treatment agent, which was mainly composed of several low temperature bacteria described in the present disclosure.

The above detailed describes the present disclosure, and is intended to make those skilled in the art being able to understand the present disclosure and thereby implement it, and should not be concluded to limit the protective scope of the present disclosure, and the present disclosure is not limited to the above-mentioned embodiments. Any equivalent variations or modifications according to the spirit of the present disclosure should be covered by the protective scope of the present disclosure.

1. A biological floc, comprising decomposed straw powder, a first microbial agent, a nitrifying bacteria and a denitrifying bacteria, wherein the first microbial agent comprises first Bacillus subtilis and selective actinomycetes.

2. The biological floc according to claim 1, wherein the first Bacillus subtilis is a combination of Bacillus subtilis KX-1 having Depository Accession Number CCTCC No. M208057, Bacillus subtilis KX-2 having Depository Accession Number CCTCC No. M208058, and Bacillus subtilis KX-4 having Depository Accession Number CCTCC No. M208060, and a mass ratio of the three is 1:(0.4-0.85):(0.5-0.85).

3. The biological floc according to claim 2, wherein the first microbial agent further comprises other beneficial microorganisms, and the other beneficial microorganisms are selected from the group consisting of Lactic acid bacteria, Bacillus natto, photosynthetic bacteria, yeasts, Bacillus licheniformis and combinations thereof, a dosage mass ratio of Bacillus subtilis KX-1 to the other beneficial microorganism is 1:(0.1-0.8).

4. The biological floc according to claim 1, wherein a dosage of the first microbial agent is 5 wt % to 25 wt % of the decomposed straw powder; and a dosage of the nitrifying bacteria and the denitrifying bacteria is equal to or greater than 80 mL per kilogram of the decomposed straw powder.

5. The biological floc according to claim 4, wherein the dosage of the first microbial agent is 8 wt % to 20 wt % of the decomposed straw powder; and the dosage of the nitrifying bacteria and the denitrifying bacteria is equal to or greater than 100 mL per kilogram of the decomposed straw powder.

6. The biological floc according to claim 1, wherein the biological floc is obtained through following steps: adding the first microbial agent to the decomposed straw powder, mixing, and then successively adding the nitrifying bacteria and the denitrifying bacteria.

7. A process for the preparation of the biological floc according to claim 1, comprising the following steps:
(1) preparing the decomposed straw powder: firstly, a second microbial agent is activated, then mixed with nutrients, and then mixed with a plurality of dry straws and a selective volcanic rock biological agent to compost and decompose the straws, and finally the straws is pulverized to >80 mesh, wherein the volcanic rock biological agent comprises a volcanic rock particle carrier and a third microbial agent implanted on the volcanic rock particle carrier, the second microbial agent and the third microbial agent respectively comprise Bacillus subtilis KX-1 having Depository Accession Number CCTCC No. M208057, actinomycetes, Bacillus subtilis KX-2 having Depository Accession Number CCTCC No. M208058, and Bacillus subtilis KX-4 having Depository Accession Number CCTCC No. M208060, a mass ratio of the four is 1:(0.3-0.75):(0.4-0.85):(0.5-0.85), the second microbial agent and the third microbial agent further selectively comprise other beneficial microorganisms and enzyme preparations, and the other beneficial microorganisms are selected from the group consisting of Lactic acid bacteria, photosynthetic bacteria, yeasts, Bacillus licheniformis and combinations thereof; and
(2) adding the first microbial agent to the decomposed straw powder, and then successively adding the nitrifying bacteria and the denitrifying bacteria to obtain the biological floc.

8. The process according to claim 7, wherein in the second microbial agent and the third microbial agent, a dosage mass ratio of Bacillus subtilis KX-1 to the other beneficial microorganism is 1:(0.1-0.8).

9. The process according to claim 7, wherein the volcanic rock biological agent is prepared through following steps:
1) grinding volcanic rocks into volcanic rock particles of 20-40 mesh, removing volcanic rock powder to obtain a volcanic rock particle carrier; and
2) preparing a bacteria solution by the third microbial agent and water according to a dosage mass ratio of 1:(20-50), then adding nitrogen source and carbon source to activate strains, and immersing the volcanic rock particle carrier in the bacteria solution for 24-72 h when more than 80% of Bacillus in the bacteria solution is in a vegetative state and viable bacteria is >5 billion/mL, and then taking out and draining the volcanic rock particle carrier to give the volcanic rock biological agent.

10. The process according to claim 7, wherein a dosage mass ratio of the second microbial agent, the volcanic rock biological agent and the dry straws is 1:(50-80):(1000-2000).

11-12. (canceled)

13. An organic bacterial fertilizer, wherein a raw material formula of the organic bacterial fertilizer comprise a sludge, the biological floc according to claim 1, and a fermenting bacteria.

14. The organic bacterial fertilizer according to claim 13, wherein the fermenting bacteria comprises a second Bacillus subtilis.

15. The organic bacterial fertilizer according to claim 14, wherein the second Bacillus subtilis is selected from the group consisting of Bacillus subtilis KX-1 having Depository Accession Number CCTCC No. M208057, Bacillus subtilis KX-2 having Depository Accession Number CCTCC No. M208058, Bacillus subtilis KX-4 having Depository Accession Number CCTCC No. M208060 and combinations thereof.

16. The organic bacterial fertilizer according to claim 15, wherein the second Bacillus subtilis is a combination of Bacillus subtilis KX-1 having Depository Accession Number CCTCC No. M208057, Bacillus subtilis KX-2 having Depository Accession Number CCTCC No. M208058, and Bacillus subtilis KX-4 having Depository Accession Number CCTCC No. M208060, and a mass ratio of the three is 1:(0.4-0.85):(0.5-0.85).

17-18. (canceled)

19. The organic bacterial fertilizer according to claim 13, wherein a dosage mass ratio of the biological floc to the sludge is 1:(1-20), and the weight ratio of the fermenting bacteria to the sludge is 0.01% to 5%.

20. The organic bacterial fertilizer according to claim 19, wherein a dosage mass ratio of the biological floc to the sludge is 1:(3-10), and the weight ratio of the fermenting bacteria to the sludge is 0.05% to 1%.

21. The organic bacterial fertilizer according to claim 13, wherein the organic bacterial fertilizer is a fermentation product obtained by mixing, composting and fermenting the sludge, the biological floc and the fermenting bacteria, or a mixture of the fermentation product and other fertilizer additives.

22. A process for the preparation of the organic bacterial fertilizer according to claim 13, comprising:
step (1) mixing and evenly stirring the biological floc, the sludge and the fermenting bacteria to form a mixed material, and stacking the mixed material into a cone, a truncated cone, a pyramid, or a truncated pyramid, covering a surface layer of the mixed material with a film and arranging an aeration pipe at a bottom of the mixed material to start composting and fermenting, and aerating inside a pile body using the aeration pipe during the composting and fermenting process;
step (2) after a core temperature of the mixed material rises up to a highest point and continues for 1-5 days, turning over the mixed material and replenishing water;
step (3) composting and fermenting the mixed material processed through said step (2) once more until fermentation is matured; and
step (4) granulating and shaping the mixed material fermented once again in said step (3), to give granular organic bacterial fertilizer.

23. The process according to claim 22, wherein the core temperature of the mixed material firstly rises up and then drops, and fermentation is matured when a difference between the core temperature and a surface temperature of the mixed material is within ±5° C.

24. The process according to claim 22, wherein said step (4) is carried out directly after said step (3), or said step (4) is carried out after mixing a product of said step (3) with other fertilizer additives.

25-26. (canceled)
